(19) United States
(12) Patent Application Publication
(10) Pub. No.: US 2021/0393720 A1
(43) Pub. Date: Dec. 23, 2021

(54) Hair Growth Compositions

(71) Applicant: Edmund Waller, Atlanta, GA (US)

(72) Inventor: Edmund Waller, Atlanta, GA (US)

(21) Appl. No.: 17/334,016

(22) Filed: May 28, 2021

Related U.S. Application Data
(60) Provisional application No. 63/031,704, filed on May 29, 2020.

(51) Int. Cl. A61K 36/185; A61K 9/107; A61K 47/44; A61K 47/46; A61K 47/10; A61K 47/02; A61P 17/14; A61K 9/00

(52) U.S. Cl. CPC A61K 36/185; A61K 9/107; A61K 47/44; A61K 47/46; A61K 2236/331; A61K 47/02; A61P 17/14; A61K 9/0014; A61K 2236/37; A61K 47/10

(57) ABSTRACT

The present application provides hair growth compositions, and more particularly compositions comprising Portulaca Oleracea, or an extract thereof, which may be useful in methods of treating hair loss and/or restoring hair color in a subject. Methods of treating hair loss and kits comprising the compositions are also provided.

### CROSS-REFERENCE TO RELATED APPLICATIONS

This application claims the benefit of U.S. Provisional Application Ser. No. 63/031,704, filed May 29, 2020, the disclosure of which is incorporated herein by reference in its entirety.

### TECHNICAL FIELD

This invention relates to hair growth compositions, and more particularly topical compositions comprising Portulaca Oleracea, or an extract thereof, which may be useful in methods of treating hair loss and/or restoring hair color in a subject.

### BACKGROUND

Hair loss is a common occurrence as individuals age or are exposed to environmental stresses including chemotherapy, radiation, toxins, infections, and auto immune disorders. Loss of hair on the scalp can cause emotional distress and problems in thermal regulation during exposure to direct sunlight in the summer or in cold weather in the winter. The most common etiology for loss of scalp hair is androgenic alopecia mediated by signaling through receptors for dihydrotestosterone receptor in the hair follicle, and leading to senescence of the hair follicle in the telogen phase with the presence of a thin, non-pigmented vellus hair. Auto-immune conditions such as alopecia areata lead to hair loss and may be treated with anti-inflammatory drugs such as inhibitors of j anus kinase (JAK), but no drug is FDA approved for this indication. Approved treatments for androgenic alopecia include minoxidil, an alpha adrenergic agonist that functions as an antihypertensive vasodilator, and finasteride, a competitive inhibitor of type II 5-alpha reductase, an enzyme involved in the biosynthesis of dihydrotestosterone.

Minoxidil (marketed for topical use as “Rogaine”) was initially developed as a blood pressure medicine and was noted to have the unexpected effect on promoting hair growth during initial clinical trials. Minoxidil is a potassium channel opener and causes hyperpolarization of cell membranes leading to vasodilation, increased blood flow, and more oxygen and nutrients to the follicles, which may be the mechanism by which it promotes hair growth. Alternatively, minoxidil may promote growth factor release by adipose-derived stem cells in the hair follicle. Minoxidil is effective in about 40% of men who experience hair regrowth after three to six months of continuous therapy and is more effective when applied to men with more recent hair loss (e.g., less than 5 years) involving smaller areas of the scalp that did not exceed four inches in diameter. Minoxidil was more effective in treating the area of baldness was located on the posterior portion of the scalp near the vertex of the head and resulted in very little or no regrowth when used to stimulate regrowth in the frontal areas of the scalp such as the anterior hairline. Thus the clinical utility of minoxidil is limited to a subset of men with androgenic alopecia, and hair regrowth in these men is limited to a portion of the scalp.

Finasteride is taken orally as a once daily pill. Finasteride blocks production of dihydrotestosterone and is therefore an antiandrogen, with reported side effects of sexual dysfunction, depression, breast enlargement, and an increased risk of prostate cancer. Current FDA approved treatments for androgenic alopecia thus have limited efficacy based upon clinical trial results and clinical practice. More effective, safe, and topical therapies for hair loss (e.g., androgenic alopecia) are therefore desirable. The present application is directed to this need and others.

### SUMMARY

The present application provides, inter alia, compositions comprising:

i) Portulaca Oleracea, or an extract thereof;

ii) a solvent comprising water and an oil component; and

iii) one or more emulsifier components.

The present application further provides methods of treating hair loss and/or restoring hair color to the original color of the hair, in a subject in need thereof, comprising administering to the subject an effective amount of a composition provided herein.

The present application further provides processes of preparing the compositions provided herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIGS. 1A-1B show a comparison of hair growth after eight months of twice daily administration of the composition of Example 1.

FIGS. 2A-2B show a comparison of hair growth after four months of twice daily administration of the composition of Example 1.

FIG. 3 shows a comparison of hair growth over fifteen months of twice daily administration of a representative hair composition of the Examples.

### DETAILED DESCRIPTION

The present invention describes a formulation to treating hair loss (e.g. androgenic alopecia) and/or restoring hair color in a subject in need thereof. An exemplary composition of the invention is provided herein, comprising an infusion of an edible herb, Portulaca Oleracea, emulsified with olive oil and beeswax. The present application describes the effectives of the representative composition in promoting conversion of hair follicles from a telogen phase with thin nonpigmented vellus hairs to an anagen phase with thicker, pigmented hairs when applied to the scalp (e.g., twice daily). The components of the compositions described herein (e.g., herbs, oils, wax, and the like) exhibit limited systemic toxicity and no apparent toxicity when applied topically. The compositions of the invention comprise biologically active drug substances that stimulate the activity of stem cells and melanocytes in the hair follicle and may have clinical applications in diseases associated with loss of regenerative stem cell activity.

Accordingly, the present application provides compositions comprising:

i) Portulaca Oleracea, or an extract thereof;

ii) a solvent comprising water and an oil component; and

iii) one or more emulsifier components.

In some embodiments, the composition provided herein is an oil-in-water composition (e.g., an oil-in-water emulsion). In some embodiments, the composition provided herein is a water-in-oil-in-water composition (e.g., a water-in-oil-in-water emulsion).

In some embodiments, the solvent further comprises an alcohol component. In some embodiments, the alcohol component comprises one or more C₁₋₆ alcohols. In some embodiments, the alcohol component comprises one or more C₁₋₃ alcohols. In some embodiments, the alcohol component comprises one or two C₁₋₃ alcohols. In some embodiments, the alcohol components comprises methanol, ethanol, or isopropanol. In some embodiments, the alcohol component comprises ethanol. In some embodiments, the alcohol component is ethanol.

In some embodiments, the present application provides compositions comprising:

i) Portulaca Oleracea, or an extract thereof;

ii) a solvent comprising water, an alcohol component, and an oil component; and

iii) one or more emulsifier components.

In some embodiments, the composition comprises Portulaca Oleracea. In some embodiments, the composition comprises an extract of Portulaca Oleracea. In some embodiments, the composition comprises a mixture of Portulaca Oleracea and an extract of Portulaca Oleracea.

In some embodiments, the solvent comprises about 0.5 to about 1.5 equivalents of water based on 1 equivalent of the oil component, for example, about 0.5, about 0.6, about 0.7, about 0.8, about 0.9, about 1.0, about 1.1, about 1.2, about 1.3, about 1.4, or about 1.5 equivalents of water based on 1 equivalent of the oil component. In some embodiments, the solvent comprises about 1 equivalent of water based on 1 equivalent of the oil component.

In some embodiments, the solvent comprises about 0.5 to about 1.5 equivalents of a mixture of the water and alcohol component, based on 1 equivalent of the oil component, for example, about 0.5, about 0.6, about 0.7, about 0.8, about 0.9, about 1.0, about 1.1, about 1.2, about 1.3, about 1.4, or about 1.5 equivalents of a mixture of the water and alcohol component based on 1 equivalent of the oil component. In some embodiments, the solvent comprises about 1 equivalent of a mixture of the water and alcohol component based on 1 equivalent of the oil component. In some embodiments, the ratio of alcohol component to water in the mixture of water and alcohol component is from about 5% to about 80% volume/volume v/v, for example, about 5% v/v, about 10% v/v, about 15% v/v, about 20% v/v, about 25% v/v, about 30% v/v, about 35% v/v, about 40% v/v, about 45% v/v, about 50% v/v, about 55% v/v, about 60% v/v, about 70% v/v, or about 80% v/v. In some embodiments, the ratio of alcohol to water in the water/alcohol mixture is about 10% v/v to about 50% v/v. In some embodiments, the ratio of alcohol to water in the water/alcohol mixture is about 20% v/v to about 40% v/v. In some embodiments, the ratio of alcohol to water in the water/alcohol mixture is about 40% v/v. In some embodiments, the ratio of alcohol to water in the water/alcohol mixture is about 20% v/v.

In some embodiments, the composition further comprises a salt component. In some embodiments, the same component comprises an alkali metal halide salt. In some embodiments, the alkali metal halide salt is an iodized salt (e.g., iodized table salt). In some embodiments, the alkali metal halide salt is selected from iodized salt, sodium chloride, and potassium chloride, or any mixture thereof. In some embodiments, the alkali metal halide salt is sodium chloride. In some embodiments, the salt component is present in mixture of water and alcohol component in a molar concentration of from about 0.05 M to about 0.15 M, for example, about 0.07 M, about 0.08 M, about 0.09 M, about 0.1 M, about 0.11 M, or about 0.12 M. In some embodiments, the salt component is present in mixture of water and alcohol component in a concentration of about 0.1 M.

In some embodiments, the solvent comprises water and an oil component selected from the group consisting of olive oil, jojoba oil, marula oil, coconut oil, canola oil, corn oil, avocado oil, sunflower oil, pomegranate oil, argan oil, rosehip oil, perilla seed oil, black seed oil, and baobab oil. In some embodiments, the solvent comprises water and olive oil. In some embodiments, the solvent comprises water and extra virgin olive oil.

In some embodiments, the composition comprises one to five emulsifier components, for example, one, two, three, four, or five emulsifier components. In some embodiments, the composition comprises one or two emulsifier components. In some embodiments, the composition comprises two emulsifier components (i.e., a first emulsifier component and a second emulsifier component).

In some embodiments, the one or more emulsifier components (e.g., a first or second emulsifier component) comprises one or more wax components. In some embodiments, the one or more emulsifier components (e.g., a first or second emulsifier component) comprises one wax component.

In some embodiments, the composition comprises about 0.05 to about 0.25 equivalents of the wax component based on 1 equivalent of the solvent, for example, about 0.05, about 0.06, about 0.07, about 0.08, about 0.09, about 0.1, about 0.11, about 0.12, about 0.13, about 0.14, about 0.15, about 0.16, about 0.17, about 0.18, about 0.19, about 0.20, about 0.21, about 0.22, about 0.23, about 0.24, or about 0.25 equivalents of the wax component based on 1 equivalent of the solvent. In some embodiments, the composition comprises about 0.1 equivalents of the wax component based on 1 equivalent of the solvent.

In some embodiments, the composition comprises about 0.15 to about 0.25 equivalents of the wax component based on 1 equivalent of the solvent, for example, about 0.15, about 0.16, about 0.17, about 0.18, about 0.19, about 0.20, about 0.21, about 0.22, about 0.23, about 0.24, or about 0.25 equivalents of the wax component based on 1 equivalent of the solvent. In some embodiments, the composition comprises about 0.2 equivalents of the wax component based on 1 equivalent of the solvent.

In some embodiments, the wax component is selected from the group consisting of beeswax, Myrica fruit wax, bayberry wax, rice bean wax, and sunflower wax. In some embodiments, the wax component is beeswax.

In some embodiments, the one or more emulsifier components (e.g., a first or second emulsifier component) comprises an emulsifier selected from the group consisting of starch, xyliance, ecomulse, wheat flour, rye flour, and bulgur flour. In some embodiments, the one or more emulsifier components (e.g., a first or second emulsifier component) comprises an emulsifier selected from the group consisting of starch, wheat flour, rye flour, and bulgur flour.

In some embodiments, the one or more emulsifier components (e.g., a first or second emulsifier component) comprises an emulsifier selected from the group consisting of xyliance, ecomulse, wheat flour, rye flour, and bulgur flour. In some embodiments, the one or more emulsifier components (e.g., a first or second emulsifier component) comprises an emulsifier selected from the group consisting of wheat flour, rye flour, and bulgur flour.

In some embodiments, the composition comprises about 0.01 to about 0.1 equivalents (e.g., by weight) of the emulsifier component based on 1 equivalent of the solvent, for example, 0.01, 0.02, 0.03, 0.04, 0.05, 0.06, 0.07, 0.08, 0.09, or 0.1 equivalents of the emulsifier component based on 1 equivalent of the solvent.

In some embodiments, the composition comprises about 0.07 to about 0.09 equivalents (e.g., by weight) of the emulsifier component based on 1 equivalent of the solvent. In some embodiments, the composition comprises about 0.08 equivalents (e.g., by weight) of the emulsifier component based on 1 equivalent of the solvent.

In some embodiments, the composition comprises about 0.01 to about 0.1 equivalents (e.g., by weight) of flour based on 1 equivalent of the solvent, for example, 0.01, 0.02, 0.03, 0.04, 0.05, 0.06, 0.07, 0.08, 0.09, or 0.1 equivalents of the flour component based on 1 equivalent of the solvent.

In some embodiments, the composition comprises about 0.07 to about 0.09 equivalents (e.g., by weight) of the flour component based on 1 equivalent of the solvent. In some embodiments, the composition comprises about 0.08 equivalents (e.g., by weight) of the flour component based on 1 equivalent of the solvent.

In some embodiments, the composition comprises about 0.001 to about 0.005 equivalents (e.g., by weight) of the emulsifier component based on 1 equivalent of the solvent, for example, 0.001, 0.002, 0.003, 0.004, or 0.005 equivalents of the emulsifier component based on 1 equivalent of the solvent. In some embodiments, the composition comprises about 0.002 equivalents (e.g., by weight) of the emulsifier component based on 1 equivalent of the solvent.

In some embodiments, the composition comprises about 0.001 to about 0.005 equivalents (e.g., by weight) of the flour based on 1 equivalent of the solvent, for example, 0.001, 0.002, 0.003, 0.004, or 0.005 equivalents of the flour based on 1 equivalent of the solvent. In some embodiments, the composition comprises about 0.002 equivalents (e.g., by weight) of the flour based on 1 equivalent of the solvent.

In some embodiments, the one or more emulsifier components (e.g., a first or second emulsifier component) comprises wheat flour. In some embodiments, the wheat flour is general purpose unbleached wheat flour.

In some embodiments, the one or more emulsifier components comprises a combination of beeswax and wheat flour (e.g., general purpose unbleached wheat flour).

In some embodiments, a composition provided herein comprises:

i) an extract of Portulaca Oleracea;

ii) a solvent comprising water and extra virgin olive oil;

iii) beeswax; and

iv) a flour component.

In some embodiments, the composition provided herein comprises:

an extract of Portulaca Oleracea;

about 0.5 to about 1.5 equivalents of water based on 1 equivalent of the extra virgin olive oil;

about 0.15 to about 0.25 equivalents of the beeswax based on 1 equivalent of the solvent; and

about 0.001 to about 0.005 equivalents of the flour component based on 1 equivalent of the solvent.

In some embodiments, the composition provided herein comprises:

an extract of Portulaca Oleracea;

about 1 equivalent of water based on 1 equivalent of the extra virgin olive oil;

about 0.2 equivalents of the beeswax based on 1 equivalent of the solvent; and

about 0.002 equivalents of the flour component based on 1 equivalent of the solvent.

In some embodiments, the composition provided herein comprises:

an extract of Portulaca Oleracea;

about 0.5 to about 1.5 equivalents of water based on 1 equivalent of the extra virgin olive oil;

about 0.1 to about 0.25 equivalents of the beeswax based on 1 equivalent of the solvent; and

about 0.001 to about 0.005 equivalents of the flour component based on 1 equivalent of the solvent.

In some embodiments, the composition provided herein comprises:

an extract of Portulaca Oleracea;

about 1 equivalent of water based on 1 equivalent of the extra virgin olive oil;

about 0.1 equivalents of the beeswax based on 1 equivalent of the solvent; and

about 0.002 equivalents of the flour component based on 1 equivalent of the solvent.

In some embodiments, the composition provided herein comprises:

i) an extract of Portulaca Oleracea;

ii) a solvent comprising water, an alcohol component, and extra virgin olive oil;

iii) beeswax; and

iv) a flour component.

In some embodiments, the composition provided herein comprises:

i) an extract of Portulaca Oleracea;

ii) a solvent comprising water, ethanol, and extra virgin olive oil;

iii) beeswax; and

iv) a flour component.

In some embodiments, the composition provided herein comprises:

an extract of Portulaca Oleracea;

a solvent comprising about 0.5 to about 1.5 equivalents of a mixture of water and an alcohol component based on 1 equivalent of the extra virgin olive oil;

about 0.1 to about 0.25 equivalents of the beeswax based on 1 equivalent of the solvent; and

about 0.001 to about 0.005 equivalents of the flour component based on 1 equivalent of the solvent. In some embodiments, the composition further comprises sodium chloride.

In some embodiments, the composition provided herein comprises:

an extract of Portulaca Oleracea;

a solvent comprising about 0.5 to about 1.5 equivalents of a 20% v/v mixture of alcohol component to water, based on 1 equivalent of the extra virgin olive oil;

about 0.1 to about 0.25 equivalents of the beeswax based on 1 equivalent of the solvent; and

about 0.001 to about 0.005 equivalents of the flour component based on 1 equivalent of the solvent. In some embodiments, the composition further comprises sodium chloride.

In some embodiments, the composition provided herein comprises:

an extract of Portulaca Oleracea;

a solvent comprising about 1 equivalent of a 20% v/v mixture of alcohol component to water, based on 1 equivalent of the extra virgin olive oil;

about 0.1 equivalents of the beeswax based on 1 equivalent of the solvent; and

about 0.002 equivalents of the flour component based on 1 equivalent of the solvent.

In some embodiments, the composition further comprises sodium chloride.

In some embodiments, the composition provided herein is an emulsion.

In some embodiments, the composition provided herein is suitable for topical administration. In some embodiments the composition provided herein is suitable for administration to the skin of the subject (e.g., head, scalp, face, neck, chest, back, arms, legs, hands, feet, and the like). In some embodiments the composition provided herein is suitable for administration to a population of hair follicles of the subject (e.g., hair follicles of the head, scalp, face, neck, chest, back, arms, legs, hands, feet, and the like).

The present application further provides processes or preparing a composition provided herein (e.g., a composition comprising Portulaca Oleracea, or an extract thereof; a solvent comprising water and an oil component; and one or more emulsifier components).

In some embodiments, the present application provides a process of preparing a composition comprising (i) Portulaca Oleracea, or an extract thereof; (ii) a solvent comprising water and an oil component; (iii) a wax component; and (iv) a flour component, the process comprising:

a) heating the Portulaca Oleracea, or an extract thereof, in the water to form an aqueous extract solution;

b) mixing the aqueous extract solution with the oil component to form an infusion mixture;

c) mixing the wax component with the oil to form an oil-wax mixture;

d) adding the infusion mixture to the oil-wax mixture to form an emulsion mixture; and

e) mixing the flour component with the emulsion mixture to form the composition.

In some embodiments, the heating of step a) comprises boiling the Portulaca Oleracea, or an extract thereof, in the water to form an aqueous extract solution.

In some embodiments, the process further comprises filtering the aqueous extract solution after the heating of step a).

In some embodiments, the process further comprises heating the oil-wax mixture of step c) at a temperature of from about 120° C. to about 150° C., for example, about 120° C. to about 140° C., about 120° C. to about 135° C., about 120° C. to about 130° C., about 120° C. to about 125° C., about 125° C. to about 140° C., about 125° C. to about 135° C., about 125° C. to about 130° C., about 130° C. to about 140° C., about 130° C. to about 135° C., or about 135° C. to about 140° C. In some embodiments, the process further comprises heating the oil-wax mixture of step c) at a temperature of from about 130° C. to about 140° C.

In some embodiments, the process further comprises cooling the oil-wax mixture to about room temperature prior to step d). As used herein, the term “room temperature” refers to a temperature that is about the temperature of the room in which the reaction is carried out, for example, a temperature from about 20° C. to about 30° C.

In some embodiments, the mixing of step e) is performed at a temperature of from about 120° C. to about 150° C., for example, about 120° C. to about 140° C., about 120° C. to about 135° C., about 120° C. to about 130° C., about 120° C. to about 125° C., about 125° C. to about 140° C., about 125° C. to about 135° C., about 125° C. to about 130° C., about 130° C. to about 140° C., about 130° C. to about 135° C., or about 135° C. to about 140° C. In some embodiments, the process further comprises heating the oil-wax mixture of step e) at a temperature of from about 130° C. to about 140° C.

In some embodiments, the present application provides a process of preparing a composition comprising (i) Portulaca Oleracea, or an extract thereof; (ii) a solvent comprising water, an alcohol component, and an oil component; (iii) a wax component; and (iv) a flour component, the process comprising:

a) heating the Portulaca Oleracea, or an extract thereof, in a mixture of the water and alcohol component (e.g., a mixture of water and ethanol), to form an alcoholic extract solution;

b) mixing the alcoholic extract solution with the oil component to form an infusion mixture;

c) mixing the wax component with the oil to form an oil-wax mixture;

d) adding the infusion mixture to the oil-wax mixture to form an emulsion mixture; and

e) mixing the flour component with the emulsion mixture to form the composition.

In some embodiments, the heating of step a) comprises boiling the Portulaca Oleracea, or an extract thereof, in the mixture of water and alcohol component (e.g., ethanol) to form an alcoholic extract solution.

In some embodiments, the process further comprises filtering the alcoholic extract solution after the heating of step a).

In some embodiments, the process further comprises heating the oil-wax mixture of step c) at a temperature of from about 120° C. to about 150° C., for example, about 120° C. to about 140° C., about 120° C. to about 135° C., about 120° C. to about 130° C., about 120° C. to about 125° C., about 125° C. to about 140° C., about 125° C. to about 135° C., about 125° C. to about 130° C., about 130° C. to about 140° C., about 130° C. to about 135° C., or about 135° C. to about 140° C. In some embodiments, the process further comprises heating the oil-wax mixture of step c) at a temperature of from about 130° C. to about 140° C. In some embodiments, the process further comprises heating the oil-wax mixture of step c) at a temperature of from about 135° C. to about 145° C. In some embodiments, the process further comprises heating the oil-wax mixture of step c) at a temperature of about 140° C.

In some embodiments, the process further comprises cooling the oil-wax mixture to about 80° C. to about 100° C. prior to step d). In some embodiments, the process further comprises cooling the oil-wax mixture to about 85° C. to about 95° C. prior to step d). In some embodiments, the process further comprises cooling the oil-wax mixture to about 90° C. prior to step d). In some embodiments, the process further comprises cooling the oil-wax mixture to about room temperature prior to step d). As used herein, the term “room temperature” refers to a temperature that is about the temperature of the room in which the reaction is carried out, for example, a temperature from about 20° C. to about 30° C.

In some embodiments, the mixing of step e) is performed at a temperature of from about 80° C. to about 100° C., for example, about 80° C. to about 95° C., about 80° C. to about 90° C., about 80° C. to about 85° C., about 85° C. to about 100° C., about 85° C. to about 95° C., about 85° C. to about 90° C., about 90° C. to about 100° C., about 90° C. to about 95° C., or about 95 C to about 100° C. In some embodiments, the mixing of step e) is performed at a temperature of from about 85° C. to about 95° C. In some embodiments, the mixing of step e) is performed at a temperature of about 90° C.

In some embodiments, the process further comprises cooling the composition to a temperature of about 0° C. or less, after the mixture of step e).

In some embodiments, the process further comprises cooling the composition to a temperature of from about −40° C. to about 10° C. after the mixture of step e), for example, about −40° C. to about 0° C., about −40° C. to about −10° C., about −40° C. to about −20° C., about −40° C. to about −30° C., about −30° C. to about 10° C., about −30° C. to about 0° C., about −30° C. to about −10° C., about −30° C. to about −10° C., about −30° C. to about −20° C., about −20° C. to about 10° C., about −20° C. to about 0° C., about −20° C. to about −10° C., about −10° C. to about 10° C., about −10° C. to about 0° C., or about 0° C. to about 10° C. after the mixing of step e).

In some embodiments, the process further comprises cooling the composition to a temperature of from about −30° C. to about 10° C. after the mixture of step e). In some embodiments, the process further comprises cooling the composition to a temperature of from about −30° C. to about −10° C. after the mixture of step e). In some embodiments, the process further comprises cooling the composition to a temperature of about −20° C. after the mixture of step e). In some embodiments, the process further comprises cooling the composition to a temperature of from about 0° C. to about 10° C. after the mixture of step e). In some embodiments, the process further comprises cooling the composition to a temperature of from about 3° C. to about 6° C. after the mixture of step e). In some embodiments, the process further comprises cooling the composition to a temperature of about 4° C. after the mixture of step e).

### Methods of Use

The present application provides methods of treating hair loss and/or restoring hair color to the original color of the hair, in a subject in need thereof. In some embodiments, the present application provides methods of treating hair loss, in a subject in need thereof. In some embodiments, the present application provides methods of restoring hair color (e.g., graying hair or white hair) to the original color of the hair in a subject in need thereof. In some embodiments, the present application provides methods of treating hair loss and restoring hair color to the original color of the hair, in a subject in need thereof. In some embodiments, the method comprises administering to the subject an effective amount (e.g., a therapeutically effective amount) of a composition provided herein. In some embodiments, the method comprises topically administering to the subject effective amount of the composition provided herein. In some embodiments, the administration comprises topical administration a localized area of the subject (e.g., a localized area of the subject affected by the hair loss such as the head, scalp, face, neck, chest, back, arms, legs, hands, feet, and the like). In some embodiments, the administration comprises topical administration to the head of the subject.

As used herein, the term “subject,” refers to any animal, including mammals. Example subjects include, but are not limited to, mice, rats, rabbits, dogs, cats, swine, cattle, sheep, horses, primates, and humans. In some embodiments, the subject is a human.

As used herein, the phrase “therapeutically effective amount” refers to the amount of composition that elicits the biological or medicinal response that is being sought in a tissue, system, animal, or subject by a researcher, veterinarian, medical doctor, or other clinician.

As used herein, the term “treating” or “treatment” can refer to one or more of (1) inhibiting the disorder (e.g., hair loss and/or hair discoloration such as graying or whitening of the hair); for example, inhibiting the disorders as described herein in an individual who is experiencing or displaying the pathology or symptomatology of the disorder (i.e., arresting further development of the pathology and/or symptomatology); and (2) ameliorating the disorder (e.g., hair loss and/or hair discoloration such as graying or whitening of the hair), for example, ameliorating the disorder in an individual who is experiencing or displaying the pathology or symptomatology of the disorder (i.e., reversing the pathology and/or symptomatology) such as decreasing the severity of the disorder.

In some embodiments, the treating comprises one or more of: increasing the thickness of the hair, increasing conversion of hair from telogen vellus hair to terminal hair shaft, increasing the density of hair follicles on the scalp, and restoring functioning melanocytes in a hair follicle, and increasing quantity of pigment of in the hair shaft.

In some embodiments, the treating comprises increasing the thickness of the hair. In some embodiments, the treating comprises increasing conversion of hair from telogen vellus hair to terminal hair shaft. In some embodiments, the treating comprises increasing the density of hair follicles on the scalp. In some embodiments, the treating comprises stimulating hair growth (e.g., increasing density of hair follicles) on the anterior portion of the scalp. In some embodiments, the treating comprises restoring functioning melanocytes in a hair follicle. In some embodiments, the treating comprises increasing quantity of pigment of in the hair shaft.

In some embodiments, the method comprises treating hair loss associated with one or more of chemotherapy, an auto-immune disorder, an androgen-mediated disorder, and aging.

In some embodiments, the method comprises treating hair loss associated with chemotherapy.

In some embodiments, the method comprises treating hair loss associated with an auto-immune disorder. In some embodiments, the auto-immune disorder is alopecia arreta.

In some embodiments, the method comprises treating hair loss associated with an androgen-mediated disorder. In some embodiments, the androgen-mediated disorder is androgenic alopecia.

In some embodiments, the method comprises treating hair loss associated with aging.

In some embodiments, the method comprises administering to the subject about 1 mL to about 10 mL of the composition, for example, about 1 mL, about 2 mL, about 3 mL, about 4 mL, about 5 mL, about 6 mL, about 7 mL, about 8 mL, about 9 mL, or about 10 mL. In some embodiments, the method comprises topically administering to the subject about 1 mL to about 10 mL of the composition. In some embodiments, the method comprises administering to the subject about 1 mL to about 5 mL of the composition. In some embodiments, the method comprises topically administering to the subject about 1 mL to about 5 mL of the composition.

In some embodiments, the method comprises administering to the subject about 2 mL to about 4 mL of the composition. In some embodiments, the method comprises topically administering to the subject about 2 mL to about 4 mL of the composition per skin surface area of 100 cm² to 300 cm².

In some embodiments, the method comprises administering to the subject about 2 mL of the composition. In some embodiments, the method comprises topically administering to the subject about 2 mL of the composition (e.g., per skin surface area of 100 cm² to 300 cm²). In some embodiments, the method comprises administering to the subject about 4 mL of the composition. In some embodiments, the method comprises topically administering to the subject about 4 mL of the composition (e.g., per skin surface area of 100 cm² to 300 cm²).

In some embodiments, the composition is administered to the subject once or twice daily. In some embodiments, the composition is administered to the subject at least twice daily (e.g., twice daily, thrice daily, and the like).

In some embodiments, the composition is administered to the subject once or twice daily. In some embodiments, the composition is administered to the subject once daily. In some embodiments, the composition is administered to the subject twice daily.

### Kits

The present application further provides kits comprising a composition described herein useful, for example, in the treatment of hair loss, which include one or more containers comprising a composition provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the composition to be administered, and/or guidelines for administration, can also be included in the kit.

### Examples

The invention will be described in greater detail by way of specific examples. The following examples are offered for illustrative purposes, and are not intended to limit the invention in any manner.

### Example 1. Representative Hair Growth Composition

50 g Portulaca Oleracea extract (i.e., Pursulane Extract; purchased from Bulk Supplements, Henderson, Nev.) was added to 250 mL of boiling water and steeped for 3 minutes. The mixture was then filtered through a paper coffee filter to remove any solid material. The resulting mixture was slowly added to 250 mL of extra virgin olive oil that was heated to 130° C.-140° C. Next, 50 g of beeswax was added to oil/water mixture, which was heated gently until all wax was dissolved. The mixture was then blended with an immersion blender (e.g., using Magic Wand® from Pro-Ver Ltd.) to form an emulsion. The initial emulsion was not stable and phase separation of liquid and oil was observed. After cooling, the mixture was re-heated to liquefy the oil/wax phase and 1 g of was flour added as an emulsifying agent. The mixture was re-blended with an immersion blender to form a stable emulsion, which was stored frozen at −20° C. in 125 mL aliquots.

### Example 2. Hair Growth Analysis

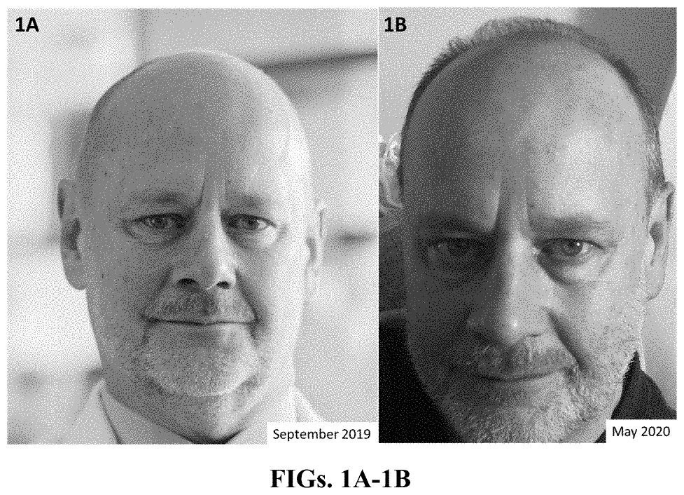

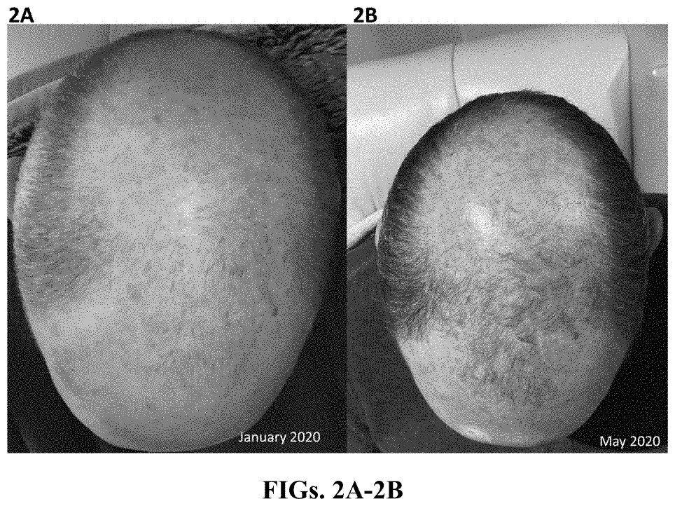

The composition of Example 1 was thawed at room temperature for use. About 2 mL of the composition was applied to the scalp and beard of a human subject, twice daily. The subject's scalp was photographed monthly to document hair growth. As shown in FIGS. 1A-2B, visual hair growth was observed over 3-9 months of twice daily administration.

### Example 3. Mechanism of Action

Without being bound by theory, it is believed that the compositions described herein (e.g., the composition of Example 1) may promote hair growth through one or more of the following mechanisms.
1. As a JAK inhibitor, e.g., functioning as an anti-inflammatory agent. Baricitinib has obtained fast-track approval from the FDA to treat alopecia arreta, an auto-immune form of alopecia (see e.g., Olamiju et al, JAAD Case Reports, 2019, 5:892-894; and Jabbari et al, EBioMedicine, 2015, 2:351-355). Other JAK inhibitors have also been reported in the treatment hair loss and/or regrowth of hair (see e.g., Jabbari et al, Exp. Dermatol. 2016, 25(8):642-643). Observation that some white hairs are converted to black hairs suggests restoration of functioning melanocytes in the hair follicle that may be due to blocking auto-immunity.
2. Via anti-androgen mechanism. Androgenic alopecia is mediated by dihydro-testosterone. The compositions described herein could have a lipophilic compound that antagonizes signaling through the testosterone receptor, similar to the action of tamoxifen with the estrogen receptor (e.g., the mechanism by which finasteride promotes hair growth in men with androgenic alopecia). See e.g., Adil et al, J. Am. Acad. Dermatol. 2017, 77(1):136-140.
3. As a hedgehog agonist that promotes stem cell activity in the hair follicle. Growth of the thickness of the hair and conversion of the hair from a telogen vellus hair into a terminal hair shaft (see e.g., Paladini et al, J. Invest. Dermatol. 2005, 125:638-646.
4. As a modulator of Dickkpof 1 (DKK-1), a pathogenic mediator involved in male pattern baldness (see e.g., Kwack et al, J. Invest. Dermatol. 2012, 132:1554-1560).
5. As a modulator of Wnt expression and/or secretion (see e.g., Li et al, J. Invest. Dermatol. 2013, 133(1):42-48; Myung et al, J. Invest. Dermatol. 2013, 133:31-41; and Lei et al, J. Invest. Dermatol. 2013, 133(1):7-9.
6. As a stimulator of autophagy (see e.g., Chai et al, Cell Reports, 2019, 27(12):3413-3421.e3).
7. As an agonist of the alpha adrenergic receptor (see e.g., Fenton et al. Br. Med. J. (Clin Res Ed), 1983, 287:1015-1017; and Olsen et al. J. Am. Acad. Dermatol. 2002, 47:377-385)

### Example 4. Alternative Manufacturing Process of a Representative Hair Growth Composition

### A. General Procedures

A representative hair growth composition was manufactured based on previously reported procedures (see e.g., The World Society of Chemistry 2017, volume 7, page 35917, the disclosure of which is incorporated herein by reference in its entirety) as an water-in-oil-in-water emulsion. In brief, water was added to oil in a ratio of 20 parts water to 80 parts oil, with a lipophilic emulsifier comprising 5% by weight of the entire mixture, which is then homogenized. The resulting oil-in-water emulsion was added to water in a ratio of 20 parts oil-in-water emulsion to 80 parts water to create a water-in-oil-in-water emulsion. The resulting emulsion was homogenized. Following homogenization, a hydrophilic emulsifier consisting of modified starch at 1% to 10% by weight of the total mixture was added.

### B. Representative Hair Growth Composition

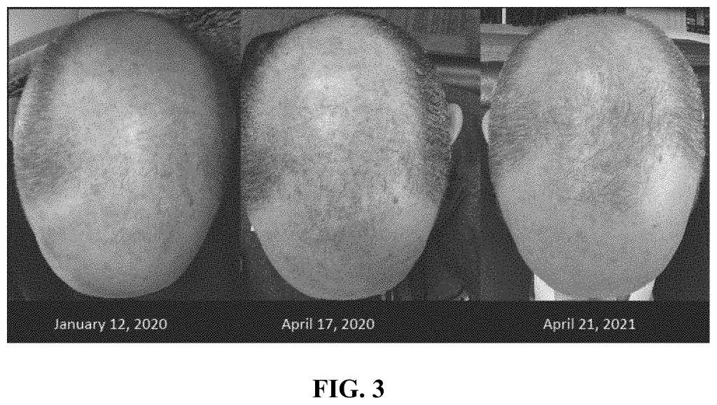

50 g of herbs was added to 125 ml of 40% v/v ethanol in water (e.g. vodka, 80 proof) and 125 mL of boiling water with ⅛ teaspoon of sodium chloride (˜0.1 M) to produce an herbal mixture in ˜0.1 M NaCl in 40% v/v ethanol/water. The herbal-water-alcohol mixture was shaken and incubated at 4° C. overnight (e.g. 12 h-18 h). The following day, the mixture was warmed to room temperature. Fifty grams of beeswax was melted and combined with 250 ml of olive oil, the resulting mixture was heated to 140° C. and then cooled to 90° C. The herbal-water-alcohol mixture was then added to the beewax/olive oil mixture stepwise with homogenization. Five portions, each of 5 g, of flour (for a total of 25 g) were added to this mixture to form an emulsion, with an additional 15 g of flour added to provide additional thickening. The resulting emulsion was then stored at −20° C. for long-term storage, or stored at 4° C. prior to administration on a twice daily basis. Longterm hair growth was visually analyzed and is shown, e.g., in FIG. 3.

Three representative lots were manufactured. Based upon photos comparing hair growth after manufacture of the first and second lots of hair composition with application for three weeks (see e.g., Example 2), the hair growth appeared to be less robust following the second lot than was apparent after the first lot. It was observed that the second lot of hair growth composition did not retain a stable emulsion and the phases separated into oil and aqueous partitions unless the composition was stored at 4° C. Without being bound by theory, it is believed that the seeming greater efficacy of the first lot may be due to slower release of active drug component from the emulsion due to a higher composition of wax and the generation of “water in oil” structures that are stabilized by wax.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

1. A composition, comprising:
i) Portulaca Oleracea, or an extract thereof;
ii) a solvent comprising water and an oil component; and
iii) one or more emulsifier components.

2. The composition of claim 1, wherein the composition comprises an extract of Portulaca Oleracea.

3. The composition of claim 1, wherein the solvent comprises about 0.5 to about 1.5 equivalents of water based on 1 equivalent of the oil component.

4. The composition of claim 1, wherein the solvent comprises about 1 equivalent of water based on 1 equivalent of the oil component.

5. The composition of claim 1, wherein the solvent comprises water and an oil component selected from the group consisting of olive oil, jojoba oil, manila oil, coconut oil, canola oil, corn oil, avocado oil, sunflower oil, pomegranate oil, argan oil, rosehip oil, perilla seed oil, black seed oil, and baobab oil.

6. The composition of claim 1, wherein the solvent comprises water and olive oil.

7. The composition of claim 1, wherein the solvent comprises water and extra virgin olive oil.

8. The composition of claim 1, wherein the composition comprises one or two emulsifier components.

9. The composition of claim 1, wherein the composition comprises a first and a second emulsifier component.

10. The composition of claim 9, wherein the first emulsifier component is a wax component.

11. The composition of claim 10, wherein the composition comprises about 0.15 to about 0.25 equivalents of the wax component based on 1 equivalent of the solvent.

12. The composition of claim 10, wherein the composition comprises about 0.2 equivalents of the wax component based on 1 equivalent of the solvent.

13. The composition of claim 10, wherein the wax component is selected from the group consisting of beeswax, Myrica fruit wax, bayberry wax, rice bean wax, and sunflower wax.

14. The composition of claim 10, wherein the wax component is beeswax.

15. The composition of claim 10, wherein the second emulsifier component is selected from the group consisting of xyliance, ecomulse, wheat flour, rye flour, and bulgur flour.

16. The composition of claim 10, wherein the second emulsifier component is wheat flour.

17. The composition of claim 1, wherein the one or more emulsifier components comprise a combination of beeswax and wheat flour.

18. The composition of claim 15, wherein the wheat flour is general purpose unbleached wheat flour.

19. The composition of claim 16, wherein the composition comprises about 0.001 to about 0.005 equivalents, by weight, of the flour based on 1 equivalent of the solvent.

20. The composition of claim 16, wherein the composition comprises about 0.002 equivalents, by weight, of the flour based on 1 equivalent of the solvent.

21. The composition of claim 1, wherein the composition comprises:
i) an extract of Portulaca Oleracea;
ii) a solvent comprising water and extra virgin olive oil;
iii) beeswax; and
iv) a flour component.

22. The composition of claim 21, wherein the composition comprises:
about 0.5 to about 1.5 equivalents of water based on 1 equivalent of the extra virgin olive oil;
about 0.15 to about 0.25 equivalents of the beeswax based on 1 equivalent of the solvent; and
about 0.001 to about 0.005 equivalents of the flour component based on 1 equivalent of the solvent.

23. The composition of claim 21, wherein the composition comprises:
about 1 equivalent of water based on 1 equivalent of the extra virgin olive oil;
about 0.2 equivalents of the beeswax based on 1 equivalent of the solvent; and
about 0.002 equivalents of the flour component based on 1 equivalent of the solvent.

24. The composition of claim 1, which is an emulsion.

25. The composition of claim 1, further comprising an alcohol component.

26. The composition of claim 25, wherein the alcohol component comprises ethanol.

27. The composition of claim 25, wherein the ratio of alcohol component to water in the composition is from about 5% to about 80% volume/volume v/v.

28. The composition of claim 25, wherein the ratio of alcohol component to water is about 20% v/v.

29. The composition of claim 1, further comprising a salt component.

30. The composition of claim 29, wherein the salt component comprises one or more alkali metal halide salts.

31. The composition of claim 29, wherein the salt component comprises sodium chloride.

32. A method of treating hair loss or in a subject in need thereof, comprising topically administering to the subject an effective amount of the composition of claim 1.

33. The method of claim 32, wherein the treating comprises one or more of: increasing the thickness of the hair, increasing conversion of hair from telogen vellus hair to terminal hair shaft, and increasing the density of hair follicles on the scalp.

34. The method of claim 32, which is a method of treating hair loss associated with one or more of chemotherapy, an auto-immune disorder, an androgen-mediated disorder, and aging.

35. The method of claim 34, wherein the auto-immune disorder is alopecia arreta.

36. The method of claim 34, wherein androgen-mediated disorder is androgenic alopecia.

37. A method of restoring hair color to the original color of the hair in a subject in need thereof, comprising topically administering to the subject an effective amount of the composition of claim 1.

38. The method of claim 37, wherein the treating comprises one or more of: restoring functioning melanocytes in a hair follicle, and increasing quantity of pigment of in the hair shaft.

39. The method of claim 32, comprising topically administering to the subject about 1 mL to about 5 mL of the composition.

40. The method of claim 32, comprising topically administering to the subject about 2 mL of the composition.

41. The method of claim 32, wherein the composition is administered to the subject at least twice daily.

42. The method of claim 32, wherein the composition is administered to the subject once or twice daily.

43. The method of claim 32, wherein the composition is topically administered to one or more of: the head, scalp, face, neck, chest, back, arms, legs, hands, and feet of the subject.

44. A process of preparing a composition comprising (i) Portulaca Oleracea, or an extract thereof; (ii) a solvent comprising water and an oil component; (iii) a wax component; and (iv) a flour component, the process comprising:
a) heating the Portulaca Oleracea, or an extract thereof, in the water to form an aqueous extract solution;
b) mixing the aqueous extract solution with the oil component to form an infusion mixture;
c) mixing the wax component with the oil to form an oil-wax mixture;
d) adding the infusion mixture to the oil-wax mixture to form an emulsion mixture; and
e) mixing the flour component with the emulsion mixture to form the composition.

45. The process of claim 44, wherein the heating of step a) comprises boiling the Portulaca Oleracea, or an extract thereof, in the water to form an aqueous extract solution.

46. The process of claim 44 or 115, further comprising filtering the aqueous extract solution after the heating of step a).

47. The process of claim 44, further comprising heating the oil-wax mixture of step c) at a temperature of from about 120° C. to about 150° C.

48. The process of claim 44, further comprising heating the oil-wax mixture of step c) at a temperature of from about 130° C. to about 140° C.

49. The process of claim 47, further comprising cooling the oil-wax mixture to about room temperature prior to step d).

50. The process of claim 44, wherein the mixing of step e) is performed at a temperature of from about 120° C. to about 150° C.

51. The process of claim 44, wherein the mixing of step e) is performed at a temperature of from about 130° C. to about 140° C.
